(19) United States
(12) Patent Application Publication
(10) Pub. No.: US 2006/0211866 A1
(43) Pub. Date: Sep. 21, 2006

(54) Process for the preparation of angiotensin receptor blockers and intermediates thereof

(75) Inventors: Narendra Shriram Joshi, Navi Mumbai (IN); Kodali Eswara Rao, Navi Mumbai (IN); Buddhavarapu Pattabhi Ramam, Navi Mumbai (IN); Shekhar Bhaskar Bhirud, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/386,010

(22) Filed: Mar. 21, 2006

Related U.S. Application Data
(60) Provisional application No. 60/667,550, filed on Apr. 1, 2005.

(30) Foreign Application Priority Data
Mar. 21, 2005 (IN) 305/MUM/2005

(51) Int. Cl. C07D 403/02; C07D 235/04

(52) U.S. Cl. 548/304.1; 548/305.4

(57) ABSTRACT

A process for the preparation of a biphenyl-containing compound of general formula I: 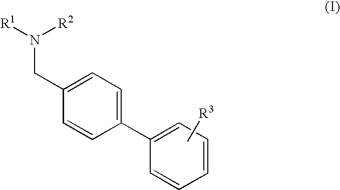
wherein R¹ is a C₃₋₆ carbonyl containing compound; R² is a substituted or unsubstituted, straight or branched C₃₋₆ alkyl group, or R¹ and R² together with the nitrogen atom to which they are bonded are joined together to form a substituted heterocyclic group selected from the group consisting of substituted or unsubstituted imidazoles, substituted or unsubstituted benzimidazoles and substituted or unsubstituted 1,3-diazaspiro[4,4]non-1-en-4-one; and R³ is a carboxylic acid ester, cyano, a substituted or unsubstituted 1H-tetrazolyl group or a substituted or unsubstituted group which may be converted in vivo into a carboxy group is provided, the process comprising reacting a compound of general formula II: 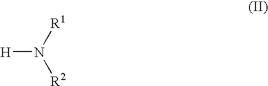
wherein R¹ and R² have the aforestated meanings with a compound of general formula III: 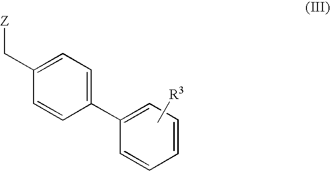
wherein Z is a leaving group and R³ has the aforestated meaning in a biphasic solvent system in the presence of a phase transfer catalyst.

### PRIORITY

This application claims the benefit under 35 U.S.C. §119 to U.S. Provisional Application No. 60/667,550, filed on Apr. 1, 2005, and entitled “PROCESS FOR THE PREPARATION OF ANGIOTENSIN RECEPTOR BLOCKERS AND INTERMEDIATES THEREOF” and to Indian Provisional Application No. 305/MUM/2005, filed on Mar. 21, 2005, and entitled “PROCESS FOR THE PREPARATION OF TELMISARTAN AND INTERMEDIATES THEREOF”, the contents of each of which are incorporated by reference herein.

### BACKGROUND OF THE INVENTION

1. Technical Field

The present invention generally relates to an improved process for the preparation of angiotensin receptor blockers (“ARBs”) and intermediates thereof.

2. Description of the Related Art

The present invention is directed to an improved process for the preparation of biphenyl-containing compounds such as, for example, ARBs and intermediates thereof. Generally, the ARBs of the present invention are angiotensin II receptor (type AT₁) antagonist. Angiotensin II is formed from angiotensin I in a reaction catalyzed by angiotensin-converting enzyme (ACE, kininase II). Angiotensin II is the principal pressor agent of the renin-angiotensin system, with effects that include vasoconstriction, stimulation of synthesis and release of aldosterone, cardiac stimulation, and renal reabsorption of sodium. ARBs block the vasoconstrictor and aldosterone-secreting effects of angiotensin II by selectively blocking the binding of angiotensin II to the AT₁ receptor in many tissues, such as vascular smooth muscle and the adrenal gland. Its action is therefore independent of the pathways for angiotensin II synthesis. ARBs are indicated for the treatment of hypertension.

Representative examples of ARBs include telmisartan, candesartan, losartan, irbesartan, and valsartan. Telmisartan (also known as 4′-[(1,4′-dimethyl-2′-propyl [2,6′-bi-1H-benzimidazol]-1′-yl)methyl]-[1,1′-biphenyl]-2-carboxylic acid) is represented by the structure of formula I. 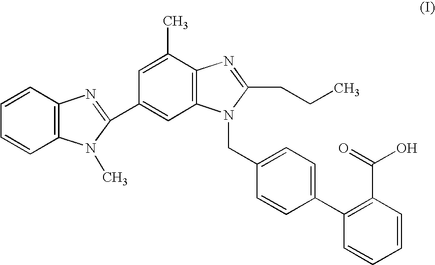
Telmisartan is sold under the trade name MICARDIS®. See, e.g., The Merck Index, Thirteenth Edition, 2001, pp. 1628-29, monograph 9209; and Physician's Desk Reference, “Micardis,” 58th Edition, pp. 1011-1013 (2004).

Candesartan (also known as (±)-1-hydroxyethyl-2-ethoxy-1-[p-(o-1H-tetrazol-5-ylphenyl)benzyl]-7-benzimidazolecarboxylate, cyclohexyl carbonate (ester)) is represented by the structure of formula II.

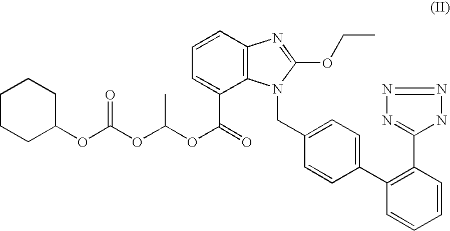

Candesartan is sold under the trade name Atacand®. See, e.g., The Merck Index, Thirteenth Edition, 2001, p. 1749, monograph 1747; and Physician's Desk Reference, “Atacand,” 58th Edition, pp. 600-602 (2004).

Losartan (also known as 2-butyl-4-chloro-1-[p-(o-1H-tetrazol-5-ylphenyl)benzyl]imidazole-5-methanol monopotassium salt) is represented by the structure of formula III. 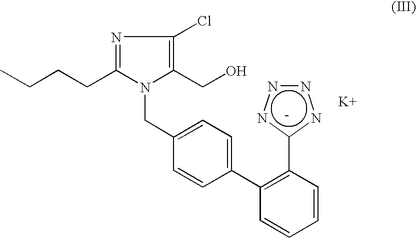
Losartan is sold under the trade name Cozaar®. See, e.g., The Merck Index, Thirteenth Edition, 2001, p. 1000, monograph 5604; and Physician's Desk Reference, “Cozaar,” 58th Edition, pp. 1952-1957 (2004).

Irbesartan (also known as 2-butyl-3-[p-(o-1H-tetrazol-5-ylphenyl)benzyl]-1,3-diazaspiro[4,4]non-1-en-4-one) is represented by the structure of formula IV. 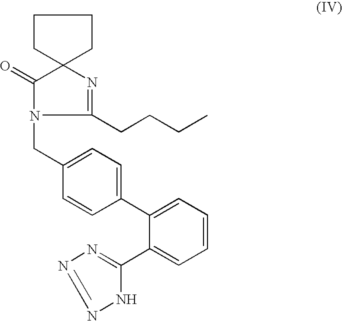
Irbesartan is sold under the trade name Avapro®. See, e.g., The Merck Index, Thirteenth Edition, 2001, p. 914, monograph 5100; and Physician's Desk Reference, “Avapro,” 58th Edition, pp. 1042-1045, 3011-3014 (2004).

Valsartan (also known as N-(1-oxopentyl)-N-[[2′-(1H-tetrazol-5-yl) [1,1′-biphenyl]-4-yl]methyl]-L-valine) is represented by the structure of formula V. 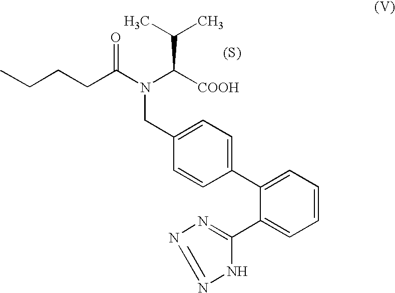
Valsartan is sold under the trade name Diovan®. See, e.g., The Merck Index, Thirteenth Edition, 2001, p. 1767, monograph 9982; and Physician's Desk Reference, “Diovan,” 58th Edition, pp. 2244-2246 (2004).

U.S. Pat. No. 5,591,762 (“the '762 patent”), herein incorporated by reference, discloses a process for preparing telmisartan. The process of the '762 patent includes reacting of 1,4′-dimethyl-2′-propyl[2,6′-bi-1H-benzimidazole] (1) with 4′-(bromomethyl)[1,1,′-biphenyl]-2-carboxylic acid 1,1-dimethylethyl ester (2) in a solvent optionally in the presence of an acid binding agent to produce the intermediate 4′-[(1,4′-dimethyl-2′-propyl[2,6′-bi-1H-benzimidazol]-1′-yl)methyl]-[1,1′-biphenyl]-2-carboxylic acid 1,1-dimethylethyl ester (3). The intermediate is further hydrolyzed to provide telmisartan (I). This reaction is generally shown below in Scheme 1.

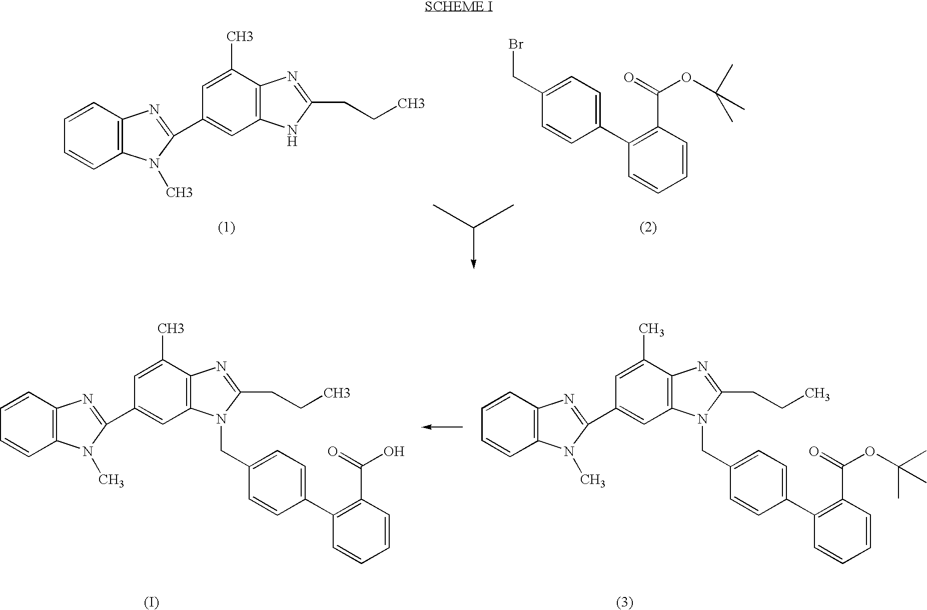

This process has been found to have several disadvantages in the commercial manufacture of a pharmaceutical. For example, this process is time consuming as column chromatography is required and consequently not as economical as the yield obtained is less and not eco-friendly as effluent generation is more.

Accordingly, there remains a need for an improved process for the preparation of ARBs and intermediates thereof that is less time consuming and is a convenient and cost efficient process that can be performed on a commercial scale.

### SUMMARY OF THE INVENTION

In accordance with one embodiment of the present invention, a process for the preparation of a biphenyl-containing compound of general formula VI: 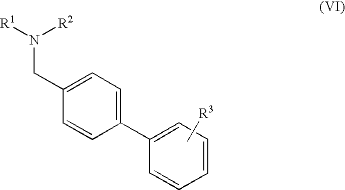
wherein R¹ is a C₃₋₆ carbonyl containing compound, R² is a substituted or unsubstituted, straight or branched C₃₋₆ alkyl group, or R¹ and R² together with the nitrogen atom to which they are bonded are joined together to form a heterocyclic group selected from the group consisting of substituted or unsubstituted imidazoles, substituted or unsubstituted benzimidazoles and substituted or unsubstituted 1,3-diazaspiro[4,4]non-1-en-4-one; and R³ is a carboxylic acid ester, cyano, a substituted or unsubstituted 1H-tetrazolyl group or a substituted or unsubstituted group which may be converted in vivo into a carboxy group, is provided, the process comprising reacting a compound of general formula VII: 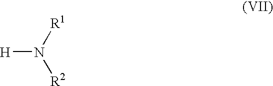
wherein R¹ and R² have the aforestated meanings with a compound of general formula VIII: 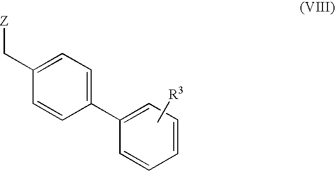
wherein Z is a leaving group and R³ has the aforestated meaning in a biphasic solvent system and in the presence of a phase transfer catalyst.

In accordance with a second embodiment of the present invention, a process for the preparation of an intermediate of telmisartan is provided, the process comprising reacting a 1,4′-dimethyl-2′-propyl[2,6′-bi-1H-benzimidazole] with a methyl 4′-(bromomethyl)[1,1 ′-biphenyl]-2-carboxylate in a biphasic solvent system in the presence of an acid binding agent and a phase transfer catalyst to provide methyl 4′-[(1,4′-dimethyl-2′-propyl[2,6′-bi-1H-benzimidazol]-1′-yl)methyl]-[1,1 ′-biphenyl]-2-carboxylate.

In accordance with a third embodiment of the present invention, a process for the purification of telmisartan is provided, the process comprising (a) providing a solution comprising telmisartan and a water-immiscible solvent; (b) treating the solution with at least one base and (c) adding an acid or a mixture of acids to the product of step (b).

The advantages of the present invention include at least:

1. Avoids the formation of any unrequired isomer by employing a biphasic reaction system.

2. Avoid the use of toxic chemicals such as potassium tert-butoxide, and tri fluoro acetic acid.

3. The resulting biphenyl-containing compound is obtained in a high yield as compared to the prior art procedure.

4. Avoids tedious purification process, e.g., column chromatography as known in the art.

5. Easy to operate on a commercial scale.

### DETAILED DESCRIPTION OF THE PREFERRED EMBODIMENTS

In general, the present invention is directed to processes for producing biphenyl-containing compounds such as, for exanple, ARBs and intermediates thereof. One embodiment of a process of the present invention involves at least reacting a compound of general formula VII: 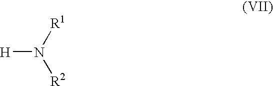
with a compound of general formula VIII: 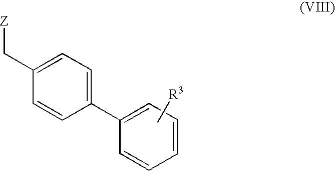
in a biphasic solvent system in the presence of a phase transfer catalyst to provide a compound of general formula VI: 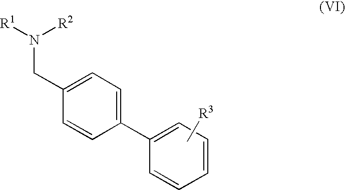
wherein R¹ is a C₃₋₆ carbonyl containing compound; R² is a substituted or unsubstituted, straight or branched C₃₋₆ alkyl group, or R¹ and R² together with the nitrogen atom to which they are bonded are joined together to form a substituted heterocyclic group selected from the group consisting of substituted or unsubstituted imidazoles, substituted or unsubstituted benzimidazoles and substituted or unsubstituted 1,3-diazaspiro[4,4]non-1-en-4-one; R3 is a carboxylic acid ester, cyano, a substituted or unsubstituted 1H-tetrazolyl group or a substituted or unsubstituted group which may be converted in vivo into a carboxy group and Z is a leaving group such as a halogen, e.g., a chlorine, bromine or iodine atom, and the like.

Representative examples of biphenyl-containing compounds which can be obtained from the process of the present invention include, but are not limited to, telmisartan, candesartan, losartan, irbesartan, valsartan and the like.

The term “phase transfer catalyst” refers to a small quantity of a chemical agent that enhances the rate of a reaction between chemical species located in different phases (immiscible liquids or solid and liquid) by extracting one of the reactants, most commonly an anion, across the interface into the other phase so that reaction can proceed. The catalyst cation is not consumed in the reaction although an anion exchange does occur. Suitable phase transfer catalysts for use herein include, but are not limited to, quaternary ammonium salts substituted with a residue such as a straight or branched alkyl group having 1 to about 18 carbon atoms, phenyl lower alkyl group including a straight or branched alkyl group having 1 to 6 carbon atoms which is substituted by an aryl group and phenyl group, e.g., tetrabutylammonium chloride, tetrabutylammonium bromide, tetrabutylammonium fluoride, tetrabutylammonium iodide, tetrabutylammonium hydroxide, tetrabutylammonium hydrogen sulfate, tributylmethylammonium chloride, tributylbenzylammonium chloride, tetrapentylammonium chloride, tetrapentylammonium bromide, tetrahexylammonium chloride, benzyldimethyloctylammonium chloride, methyltrihexylammonium chloride, benzylmethyloctadecanylammonium chloride, methyltridecanylammonium chloride, benzyltripropylammonium chloride, benzyltriethylammonium chloride, phenyltriethylammonium chloride, tetraethylammonium chloride, tetramethylammonium chloride and the like; phosphonium salts substituted with a residue such as a straight or branched alkyl group having 1 to about 18 carbon atoms, e.g., tetrabutylphosphonium chloride and the like; and pyridinium salts substituted with a straight or branched alkyl group having 1 to about 18 carbon atoms, e.g., 1-dodecanylpyridinium chloride and the like.

Among these phase transfer catalysts, quaternary ammonium salts substituted with a straight or branched alkyl group having 1 to about 18 carbon atoms such as tetrabutylammonium chloride and the like are particularly preferred. As the salt-forming ions in these salts, hydroxyl ion, hydrogen sulfate ion and halogen ions are preferred, among which the chlorine ion is particularly preferred. If desired, sodium sulfite or the like may be added to the reaction system of the above-mentioned reaction for the purpose of preventing the coloration caused by oxidation. tetrabutyl ammonium bromide, tetrabutyl ammonium hydroxide, tetramethyl ammonium iodide, tetrabutyl ammonium sulfate and the like and mixtures thereof. Generally, the amount of the phase transfer catalyst employed can range from about 1% w/w to about 10% w/w and preferably from about 5% w/w to about 10% w/w.

The coupling of the compound of formula VII with the compound of formula VIII is advantageously carried out in a biphasic solvent system. Generally, a biphasic solvent system includes at least an aqueous phase and a water-immiscible phase, e.g., water and a water-immiscible solvent. Water-immiscible solvents are generally those solvents which when allowed to stand and remain undisturbed, after being initially mixed with water, separate out into a distinct layer different from the water layer. The two layers (or phases) are typically visible to the naked eye. Suitable water-immiscible solvents include, but are not limited to, halogenated hydrocarbon solvents, aromatic hydrocarbon solvents, aliphatic hydrocarbon solvents, ketone solvents, ether solvents, and the like and mixtures thereof. Exemplary water-immiscible solvents include, but are not limited to, methylene chloride toluene, methyl isobutyl ketone, tert-butyl methyl ether, heptane, cyclohexane, n-heptane, hexane, octanol, n-decane, decalene and mixtures thereof.

The temperature of the reaction will ordinarily range from about 20° C to about 50° C. The time period for the reaction can range from about 2 hours to about 10 hours. The compound of formula VIII will ordinarily be present in an amount ranging from about 1 to about 1.5 moles per mole of the compound of formula VII and preferably from about 1 to about 1.1 moles per mole of the compound of formula VII.

If desired, the reaction can be carried out in the presence of an acid binding agent. Suitable acid binding agents include, but are not limited to, alkali metal hydroxide, e.g., sodium hydroxide and potassium hydroxide, alkali metal carbonate, e.g., sodium carbonate, and the like and mixtures thereof. Generally, the acid binding agent can be employed in suitable amounts as can be determined by one skilled in the art. For example, the acid binding agent can be present in an amount ranging from about 1 molar equivalent to about 5 molar equivalent per equivalent of the compound of formula VIII.

In one embodiment, a process of the present invention produces an intermediate of telmisartan which includes at least reacting 1,4′-dimethyl-2′-propyl[2,6′-bi-1H-benzimidazole] (1) with methyl 4′-(bromomethyl)[1,1′-biphenyl]-2-carboxylate (7) in a biphasic solvent system in the presence of an acid binding agent and a phase transfer catalyst to provide intermediate methyl 4′-[(1,4′-dimethyl-2′-propyl[2,6′-bi-1H-benzimidazol]-1′-yl)methyl]-[1,1′-biphenyl]-2-carboxylate (8) as generally shown below in Scheme II:

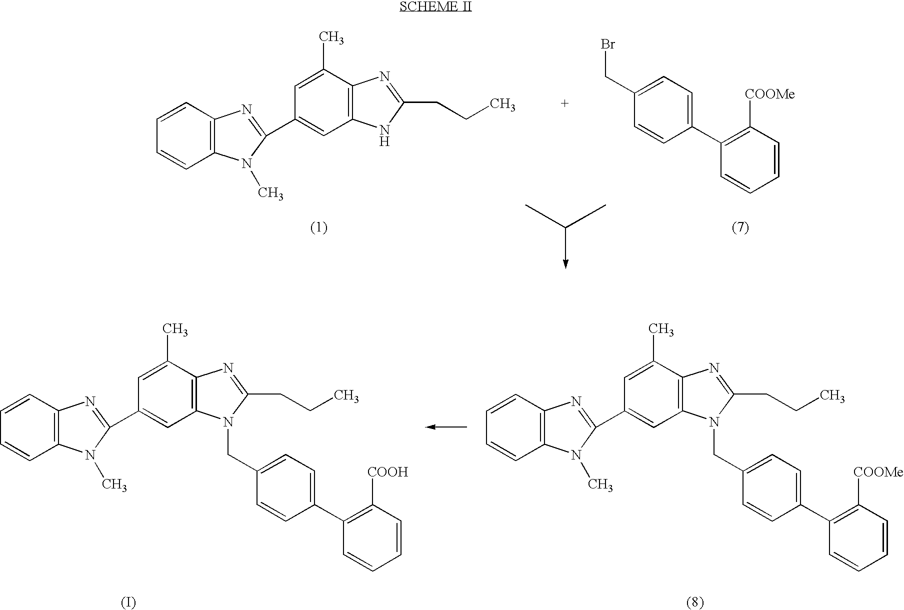

Generally, following the formation of the intermediate of formula 8, the intermediate can then be hydrolyzed to provide telmisartan. The hydrolysis can be carried out in the presence of a base such as, for example, an alkali metal hydroxide or carbonate, e.g., sodium hydroxide, potassium hydroxide, potassium carbonate, etc., and in a suitable solvent such as water, an alcohol, e.g., methanol, ethanol, isopropanol, and the like, and mixtures thereof, e.g., water/methanol. The temperature employed during hydrolysis can range from about 50° C to boiling temperature of the reaction mass.

The biphenyl-containing compounds obtained herein can also be purified. For example, crude telmisartan can be purified in one or more solvents such as a halogenated hydrocarbon solvent, e.g., methylene chloride and the like; a lower alcohol, e.g., methanol, ethanol, isopropanol and the like; amides, e.g., N,N-dimethylformamide, N,N-dimethylacetamide and the like; and mixtures thereof. The process of the present invention advantageously provides biphenyl-containing compounds in relatively high purity, e.g., equal to or greater than about 98%, preferably equal to or greater than about 99% and most preferably equal to or greater than about 99.5% as determined by HPLC (“High Pressure Liquid Chromatography”).

In one embodiment, telmisartan can be purified by at least dissolving telmisartan in a water-immiscible solvent follow by the addition of a suitable base and then the addition of a suitable acid. A suitable water-immiscible solvent can be a lower alcohol such as methanol, ethanol, 2-propanol, and the like and mixtures thereof. A suitable base includes, but is not limited to, a nitrogen-containing base, e.g., ammonia (gas or aqueous solution), triethyl amine, diisopropyl amine, dimethyl amine, monomethyl amine (gas or aqueous solution) diisopropyl ethyl amine and the like; an alkali metal hydroxide, e.g., sodium hydroxide, potassium hydroxide and the like; a sodium alcoholate, e.g., sodium methoxide, sodium ethoxide, sodium isopropxide and the like and mixtures thereof. Suitable acids include, but are not limited to, acetic acid, hydrochloric acid, hydrobromic acid, sulfuric acid and the like and mixtures thereof. The process of the present invention advantageously provides telmisartan in relatively high purity, e.g., equal to or greater than about 98%, preferably equal to or greater than about 99% and most preferably equal to or greater than about 99.5% as determined by HPLC.

If desired, the biphenyl-containing compounds obtained herein such as telmisartan can thereafter be converted to a pharmaceutically acceptable salt, e.g., telmisartan hydrochloride, by techniques known in the art. For example, after preparing the compound of Formula I corresponding to losartan, the compound Formula I will be further reacted with a suitable source of potassium to provide the potassium salt. The reaction to prepare the potassium salt is known to those skilled in the art.

Another aspect of the present invention is directed to pharmaceutical compositions containing the biphenyl-containing compounds or pharmaceutically acceptable salts thereof, particularly telmisartan, candesartan, losartan, irbesartan, and valsartan. In addition to the active ingredient(s), the pharmaceutical compositions of the present invention can contain one or more pharmaceutically acceptable excipients. The selection of excipients and the amounts to use can be readily determined by the formulation scientist based upon experience and consideration of standard procedures and reference works in the field. For example, the most recent edition of Handbook of Pharmaceutical Excipients, American Pharmaceutical Association, can be used as a guidance.

The following examples are provided to enable one skilled in the art to practice the invention and are merely illustrative of the invention. The examples should not be read as limiting the scope of the invention as defined in the claims.

### EXAMPLE 1

Preparation of Methyl 4′-[(1,4′-dimethyl-2′-propyl[2,6′-bi-1H-benzimidazol]-1′-yl)methyl]-[1,1 ′-biphenyl]-2-carboxylate

1,4′-dimethyl-2′-propyl[2,6′-bi-1H-benzimidazole] (40 g) in methyl isobutyl ketone (“MIBK”, 160 ml) was placed in a vessel and a potassium hydroxide solution (36.8 g KOH in 200 ml water) was added. Methyl 4′-(bromomethyl)[1,1′-biphenyl]-2-carboxylate (44 g) and tert-butyl ammonium bromide (4 g) were added. The reaction mixture was stirred for 2 hours at 25 to 30° C. The reaction mixture was filtered and washed with MIBK (40 ml).

Dry the material at 60 to 65° C.

Dry wt. 78 grams

### EXAMPLE 2

Preparation of Telmisartan

The methyl 4′-[(1,4′-dimethyl-2′-propyl[2,6′-bi-1H-benzimidazol]-1′-yl)methyl]-[1,1′-biphenyl]-2-carboxylate (45 g) obtained in Example 1 in methanol (225 ml) were charged to a reaction vessel. A potassium hydroxide solution (19 g KOH in 55 ml water) was added and the reaction mass was heated to reflux for 3 hours. The reaction mass was cooled to 25° C. and the pH was adjusted to 6 using dilute HCl. The solid obtained was filtered and dried (37 g). The dry solid was taken in a combination of dichloromethane and methanol (8 volumes to 2 volumes). The insolubles were filtered and the reaction mass was concentrated in methylene dichloride and isolated the solid from methanol.

Dry wt. 32 grams

### EXAMPLE 3

Purification of Crude Telmisartan

Crude telmisartan (30 grams) was dissolved in N, N-dimethyl formamide (300 ml) at 85 to 90° C. under stirring. The reaction mixture was allowed to cool to room temperature. The solid obtained was filtered and dried at 60 to 65° C. The purity of telmisartan was greater than 99.5 % as determined by HPLC.

### EXAMPLE 4

Purification of Telmisartan

Telmisartan (20 grams) was dissolved in methanol (100 ml) and followed by the addition of 4 ml of 25% aq. ammonia at 25 to 30° C. The solution was then stirred for 30 minutes. Next, the solution was filtered through a hyflo bed, washed with methanol (20 ml), and then neutralized with acetic acid (2.6 ml) by adjusting pH from 6 to 7. The neutralized solution was stirred for 30 minutes and 80-100 ml of distilled water was added in the reaction mixture. The reaction mixture was further stirred for 2 hours at 25 to 30° C. The resulting solid was filtered and washed with purified water (40 ml) and methanol (40 ml). The solid obtained was dried at 60 to 65° C. under vacuum. HPLC Purity-99.6%

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A process for the preparation of a biphenyl-containing compound of general formula I: 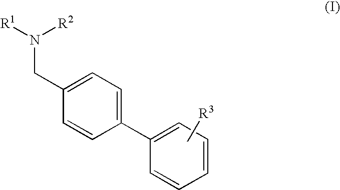
wherein R¹is a C₃₋₆ carbonyl containing compound; R² is a substituted or unsubstituted, straight or branched C₃₋₆ alkyl group, or R¹ and R² together with the nitrogen atom to which they are bonded are joined together to form a substituted heterocyclic group selected from the group consisting of substituted or unsubstituted imidazoles, substituted or unsubstituted benzimidazoles and substituted or unsubstituted 1,3-diazaspiro[4,4]non-1-en-4-one; and R³ is a carboxylic acid ester, cyano, a substituted or unsubstituted 1H-tetrazolyl group or a substituted or unsubstituted group which may be converted in vivo into a carboxy group, the process comprising reacting a compound of general formula II: 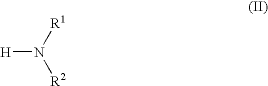
wherein R¹ and R² have the aforestated meanings with a compound of general formula III: 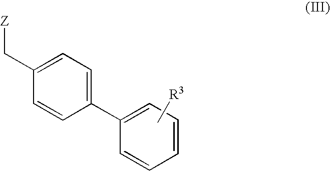
wherein Z is a leaving group and R³ has the aforestated meaning in a biphasic solvent system and in the presence of a phase transfer catalyst.

2. The process of claim 1, wherein the biphasic solvent system comprises water and a water immiscible solvent.

3. The process of claim 2, wherein the water immiscible solvent is selected from the group consisting of a halogenated hydrocarbon, aromatic hydrocarbon, aliphatic hydrocarbon, ketone, ether, and mixtures thereof.

4. The process of claim 2, wherein the water immiscible solvent is selected from the group consisting of methylene chloride, toluene, methyl isobutyl ketone, tert-butyl methyl ether, heptane, cyclohexane, n-heptane, hexane, octanol, n-decane, decalene and mixtures thereof.

5. The process of claim 1, wherein the phase transfer catalyst is selected from the group consisting of a quaternary ammonium phase transfer catalyst, a phosphonium phase transfer catalyst and a pyridinium phase transfer catalyst.

6. The process of claim 1, wherein the phase transfer catalyst is a quaternary ammonium phase transfer catalyst selected from the group consisting of tricaprylylmethylammonium chloride, tetra-n-butylammonium bromide, benzyltriethylammonium chloride, cetyltrimethylammonium bromide, cetylpyridinium bromide, N-benzylquininium chloride, tetra-n-butylammonium chloride, tetra-n-butylammonium hydroxide, tetra-n-butylammonium iodide, tetra-ethylammonium chloride, benzyltributylammonium bromide, benzyltriethylammonium bromide, hexadecyltriethylammonium chloride, tetramethylammonium chloride, hexadecyltrimethyl ammonium chloride, octyltrimethylammonium chloride and mixtures thereof.

7. The process of claim 1, wherein the phase transfer catalyst is selected from the group consisting of tetrabutyl ammonium bromide, tetrabutyl ammonium hydroxide, tetramethyl ammonium iodide, tetrabutyl ammonium sulfate and mixtures thereof.

8. The process of claim 1, wherein the reaction is carried out in the presence of an acid binding agent.

9. The process of claim 8, wherein the acid binding agent is selected from the group consisting of an alkali metal hydroxide, alkali metal carbonate, alkali metal bicarbonate and mixtures thereof.

10. The process of claim 8, wherein the acid binding agent is selected from the group consisting of sodium hydroxide, potassium hydroxide, sodium carbonate and mixtures thereof.

11. The process of claim 1, wherein the compound of formula I is methyl 4′-[(1,4′-dimethyl-2′-propyl[2,6′-bi-1H-benzimidazol]-1′-yl)methyl]-[1,1′-biphenyl]-2-carboxylate.

12. The process of claim 11, wherein the methyl 4′-[(1,4′-dimethyl-2′-propyl[2,6′-bi-1H-benzimidazol]-1′-yl)methyl]-[1,1′-biphenyl]-2-carboxylate is thereafter converted to telmisartan or a pharmaceutically acceptable salt thereof.

13. The process of claim 11, further comprising hydrolyzing the methyl 4′-[(1,4′-dimethyl-2′-propyl[2,6′-bi-1H-benzimidazol]-1′-yl)methyl]-[1,1′-biphenyl]-2-carboxylate to provide telmisartan.

14. The process of claim 13, wherein the step of hydrolyzing is carried out in the presence of a base and in a solvent.

15. The process of claim 14, wherein the base is selected from the group consisting of sodium hydroxide, potassium hydroxide, potassium carbonate and mixtures thereof.

16. The process of claim 14, wherein the solvent is selected from the group consisting of water, methanol, ethanol, isopropanol, and mixtures thereof.

17. The process of claim 12, further comprising purifying telmisartan or a pharmaceutically acceptable salt thereof.

18. The process of claim 17, wherein the telmisartan is purified in a solvent selected from the group consisting of a halogenated hydrocarbon, alcohol, and mixtures thereof.

19. Telmisartan prepared in accordance with the process of claim 1 and having a purity of equal to or greater than about 98%.

20. The process of claim 1, wherein in the compound of formula I R¹ and R² together with the nitrogen to which they are bonded represent a substituted benzimidazole and R³ is a substituted or unsubstituted 1H-tetrazolyl group.

21. The process of claim 20, wherein the compound of formula I is candesartan of the formula

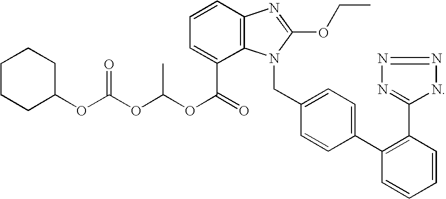

22. The process of claim 1, wherein in the compound of formula I R¹ and R² together with the nitrogen to which they are bonded represent a substituted imidazole and R³ is a substituted or unsubstituted 1H-tetrazolyl group.

23. The process of claim 22, wherein the compound of formula I is thereafter converted to a pharmaceutically acceptable salt thereof.

24. The process of claim 22, wherein the compound of formula I is thereafter converted to a potassium salt.

25. The process of claim 24, wherein the compound is of the formula

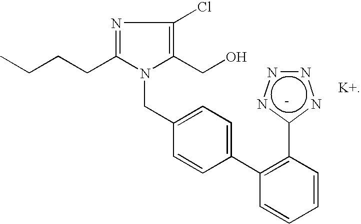

26. The process of claim 1, wherein in the compound of formula I R¹ and R² together with the nitrogen to which they are bonded represent a substituted 1,3-diazaspiro[4,4]non-1-en-4-one and R³ is a substituted or unsuubstituted 1H-tetrazolyl group.

27. The process of claim 26, wherein the compound of formula I is irbesartan of the formula

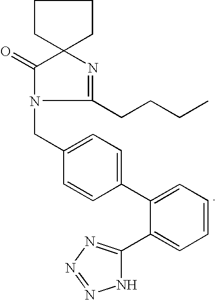

28. The process of claim 1, wherein in the compound of formula I R¹ is a C₅ carbonyl containing compound, R² is a C₃-C₆ substituted alkyl group and R³ is a substituted or unsubstituted 1H-tetrazolyl group.

29. The process of claim 28, wherein the compound of formula I is valsartan of the formula

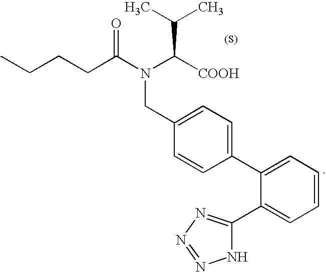

30. A pharmaceutical composition comprising the compound obtained from the process of claim 1 and at least one pharmaceutically acceptable excipient.

31. A process for preparing an intermediate of telmisartan, the process comprising reacting a 1,4′-dimethyl-2′-propyl[2,6′-bi-1H-benzimidazole] with a methyl 4′-(bromomethyl)[1,1′-biphenyl]-2-carboxylate in a biphasic solvent system in the presence of an acid binding agent and a phase transfer catalyst to provide methyl 4′-[(1,4′-dimethyl-2′-propyl[2,6′-bi-1H-benzimidazol]-1′-yl)methyl]-[1,1′-biphenyl]-2-carboxylate.

32. The process of claim 31, further comprising hydrolyzing the methyl 4′-[(1,4′-dimethyl-2′-propyl[2,6′-bi-1H-benzimidazol]-1′-yl)methyl]-[1,1′-biphenyl]-2-carboxylate to provide telmisartan.

33. The process of claim 32, further comprising purifying telmisartan or a pharmaceutically acceptable salt thereof.

34. The process of claim 33, wherein the purity of telmisartan is equal to or greater than about 98%.

35. A process for the purification of telmisartan comprising (a) providing a solution comprising telmisartan and a water-immiscible solvent; (b) treating the solution with at least one base and (c) adding an acid or a mixture of acids to the product of step (b).

36. The process of claim 35, wherein the water-immiscible solvent is a lower alcohol, the base is selected from the group consisting of a nitrogen-containing base, alkali metal hydroxide, sodium alcoholate and mixtures thereof and the acid is selected from the group consisting of acetic acid, hydrochloric acid, hydrobromic acid, sulfuric acid and mixtures thereof.

37. The process of claim 35, wherein the water-immiscible solvent is selected from the group consisting of methanol, ethanol, 2-propanol, and mixtures thereof, the base is selected from the group consisting of ammonia, triethyl amine, diisopropyl amine, dimethyl amine, monomethyl amine, diisopropyl ethyl amine and mixtures thereof, and the acid is selected from the group consisting of acetic acid, hydrochloric acid, hydrobromic acid, sulfuric acid and mixtures thereof.

38. The process of claim 37, wherein the telmisartan has a purity equal to or greater than about 99.5%.
